(19) United States
(12) Patent Application Publication
(10) Pub. No.: US 2024/0323169 A1
(43) Pub. Date: Sep. 26, 2024

(54) SYSTEM AND DEVICE FOR DISPLAY SHARING

(71) Applicant: Reachora Limited, London (GB)

(72) Inventors: Justin DAVIES, Monaco (MC); Max PETERS, London (GB); Adam SNOW, London (GB)

(21) Appl. No.: 18/189,117

(22) Filed: Mar. 23, 2023

(51) Int. Cl. H04L 9/40; G16H 40/67; H04L 67/12

(52) U.S. Cl. CPC H04L 63/0428; G16H 40/67; H04L 67/12

(57) ABSTRACT

A system for sharing visual data from a medical device includes: a first user terminal configured to obtain medical device visual data, a sharing device connected to the first user terminal, configured to receive the medical device visual data from the first user terminal and communicate it to a server, and a remote user device. The sharing device is operable to receive a command to share the medical device visual data, and in response to receiving that command, to communicate the medical device visual data to the server. The remote user device is operable to receive a communication from the server including the medical device visual data, and to display the medical device visual data via its respective display.

### FIELD

The present disclosure relates to systems and devices for sharing visual information. Such systems and devices as described are particularly suited for sharing visual information relating to medical systems and devices over a remote connection.

### BACKGROUND

Within the medical system, multiple establishments such as hospitals, specialist medical clinics and general practitioner surgeries, have a need to share expertise and data. A medical scan may be taken in one facility, and the results of the scan recorded to a patient file, which may then be sent to or accessed from another part of the facility or from a different facility.

For example, a general practitioner may meet with a patient in person to diagnose a condition, and determine that the patient should be referred to a specialist clinic. At the clinic, the patient may be referred for a scan procedure, the results of which are then reviewed at a later appointment, at which a medical consultant assesses the results of the scan. It may be the case that the patient's condition can be diagnosed accurately at that stage by the consultant. Alternatively, the consultant may require further expert medical input from a specialist in the field.

The number of medical specialists in each field of medicine is relatively limited compared to the number of patients being seen, and the number of medical and scanning facilities. It is not usual that a specialist is present in the scan appointment, for example, and so irregularities in the scan results or questions from medical personnel arising at that stage may not be answered until a later time; by a consultant in the clinic as the results are reviewed, or by a further specialist following that consultation.

The delay in obtaining expert medical input at an early stage, on issues arising during the consultation and scan process for example, may lead to inefficiencies in the medical system. For example, it may be deemed necessary to repeat a scan, which in turn involves additional administrative tasks in arranging the appointment, and additional costs both in terms of monetary costs and the time of the medical professionals involved. Further, there is a clear detriment to the patient in having to rearrange and participate in further scans and appointments, as well as the delay to diagnosis and treatment. All of these factors contribute to a negative impact on the outcomes for the patient, and on the efficiency of the system as a whole.

The present disclosure aims to overcome or reduce the problems associated with the state of the art.

### BRIEF DESCRIPTION OF THE DISCLOSURE

According to a first aspect of the disclosure, we provide a system for sharing visual data from a medical device, the system including: a first user terminal configured to obtain medical device visual data, the first user terminal providing a first user display configured to display the medical device visual data, a sharing device connected to the first user terminal, and providing a communication module configured to communicate with a server, the sharing device being configured to receive the medical device visual data from the first user terminal and a remote user device providing a display, and a respective communication module configured to communicate with the server, wherein the sharing device is operable to receive a command to share the medical device visual data, and in response to receiving that command, to communicate the medical device visual data to the server, and wherein the remote user device is operable to receive a communication from the server including the medical device visual data, and to display the medical device visual data via its respective display.

The sharing device may be configured to encrypt the medical device visual data, and to communicate the encrypted medical device visual data to the server.

The server may be configured to communicate the encrypted medical device visual data to the remote user device, and the remote user device may be configured to decrypt the medical device visual data.

The medical device may be a medical imaging device comprising one or more of: a radiographic imaging device, a magnetic resonance imaging device, a computerised tomography scanner, a positron emission tomography scanner, an ultrasound scanner, a photoacoustic imaging scanner, a functional near-infrared spectroscopy scanner, a magnetic particle imaging scanner.

The first user terminal may comprise a first processing device connected to the medical device for operating the medical device and receiving data from the medical device, the data including the medical device visual data, wherein the first processing device is connected to the sharing device via a display interface.

The sharing device may be connected to the first user display via a sharing device display interface, such that the medical device display data is routed to the first display device from the first processing device via the sharing device.

The sharing device may provide a user-operable actuator via which the command to share the medical device visual data is provided to the sharing device.

The system may further include a local user device having a processor and communication module, the local user device being operable to initiate a communication channel with the remote user device via the server, wherein the local user device is further operable to initiate sharing of the medical device visual data by causing the server to communicate the command to share the medical device visual data to the sharing device, and the server provides the medical device visual data to the remote user device.

The first user terminal may be operable to initiate a communication channel with the remote user device via the server, wherein the first user terminal is further operable to initiate sharing of the medical device visual data by causing the server to communicate the command to share the medical device visual data to the sharing device, and the server provides the medical device visual data to the remote user device.

The system may be configured such that the server stores data including a record associating an identifier of the sharing device and a communication address of the sharing device, the server being configured to receive a request to share medical device visual data, the request specifying an identifier relating to the sharing device, and in response, to locate the record matching the specified identifier, and to communicate with the sharing device at the corresponding communication address so as to provide the command to share the medical device visual data.

According to a second aspect of the disclosure, we provide a sharing device for use in the system of the first aspect of the disclosure, the sharing device including: a processor, a port configured to receive medical device visual data from a display interface of a first user terminal, and a communication module configured to communicate with a server, configured such that on receipt of a command to share the medical device visual data, the medical device visual data is communicated to the server.

The sharing device may provide an encryption module configured to encrypt the medical device visual data, and the sharing device being configured to communicate the encrypted medical device visual data to the server.

The sharing device may further include a mechanical actuator configured for operation by a user, such that operation of the actuator provides the command to share the medical device visual data.

The sharing device may be configured to receive a command to share the medical device visual data from the server via the communication module.

According to a third aspect of the disclosure, we provide a computer-implemented method for sharing visual data from a medical device, the method including the steps of: obtaining at a first terminal medical device visual data, communicating the medical device visual data via a display interface to a sharing device, receiving at the sharing device a command to share the medical device visual data, in response to receiving that command, communicating the medical device visual data from the sharing device to a server, communicating the medical device visual data, from the server to a remote user device, and displaying the medical device visual data via a display of the remote user device.

The method may further include encrypting the medical device visual data at the sharing device for communication to the server, and subsequently decrypting the encrypted medical device visual data at the remote user device.

The method may further include the steps of: at a local user device, initiating a communication channel with the remote user device via the server, and at the local user device, communicating to the server a request to share medical device visual data, the request specifying an identifier relating to the sharing device, receiving at the server the request to share medical device visual data, locating at the server a stored record matching the specified identifier and an associated communication address of the sharing device, and using the communication address to communicate to the sharing device the command to share the medical device visual data.

According to a fourth aspect of the disclosure, we provide a computer-implemented method for sharing visual data from a medical device, performed at a server, the method including the steps of: storing multiple data each including a record associating an identifier of one of multiple sharing devices with a communication address for accessing that respective sharing device; receiving a communication from a first user device to initiate a communication channel with a specified second user device, communicating with the specified second user device to initiate the communication channel between the first and second user devices, receiving from the first user device a request to share medical device visual data, the request specifying an identifier relating to one of the multiple sharing devices, and in response, locating the record matching the specified identifier, sending a command to share medical device visual data to the sharing device at the communication address stored in the located record, receiving from the sharing device medical device visual data, and communicating the medical device visual data to the second user device.

According to a fifth aspect of the disclosure we provide a computer program providing instructions which, when the program is executed by a computer, cause the computer to carry out the steps of: storing multiple data each including a record associating an identifier of one of multiple sharing devices with a communication address for accessing that respective sharing device; receiving a communication from a first user device to initiate a communication channel with a specified second user device, communicating with the specified second user device to initiate the communication channel between the first and second user devices, receiving from the first user device a request to share medical device visual data, the request specifying an identifier relating to one of the multiple sharing devices, and in response, locating the record matching the specified identifier, sending a command to share medical device visual data to the sharing device at the communication address stored in the located record, receiving from the sharing device medical device visual data, and communicating the medical device visual data to the second user device.

According to a sixth aspect of the disclosure we provide a computer-readable medium providing instructions which, when executed by a computer, cause the computer to carry out the steps of: storing multiple data each including a record associating an identifier of one of multiple sharing devices with a communication address for accessing that respective sharing device; receiving a communication from a first user device to initiate a communication channel with a specified second user device, communicating with the specified second user device to initiate the communication channel between the first and second user devices, receiving from the first user device a request to share medical device visual data, the request specifying an identifier relating to one of the multiple sharing devices, and in response, locating the record matching the specified identifier, sending a command to share medical device visual data to the sharing device at the communication address stored in the located record, receiving from the sharing device medical device visual data, and communicating the medical device visual data to the second user device.

### BRIEF DESCRIPTION OF THE FIGURES

In order that the present disclosure may be more readily understood, preferable embodiments will now be described, by way of example only, with reference to the accompanying drawings, in which:

FIG. 1 is a diagrammatic representation of the system according to embodiments of the described technology;

FIG. 2 is a diagrammatic representation of a sharing device according to embodiments of the described technology;

FIG. 3 is a diagrammatic representation of a first user terminal including a first user display and a first processing device, connected to a medical device and other peripherals, and connected to a sharing device via a display interface of the first processing device;

FIGS. 4A and 4B are diagrammatic representations of a local user device and a remote user device, respectively;

FIG. 5 is an exemplary communication channel interface displayed on a display of a device;

FIG. 6 is a diagrammatic representation of a server for use in the system according to embodiments of the described technology;

FIGS. 7 and 8 are a diagrammatic representation of a system according to embodiments of the described technology in which multiple sharing devices and multiple remote devices are provided, and a flow of communications within the system during an exemplary use case, respectively;

FIG. 9 is a flow chart illustrating steps involved in sharing medical device visual data from the sharing device with a remote user, according to embodiments of the described technology;

FIG. 10 is a flow chart illustrating the steps involved in a local user device initiating sharing the medical device visual data, according to embodiments of the described technology; and

FIG. 11 is a flow chart illustrating the steps involved in operating the server of the system according to embodiments of the described technology.

### DETAILED DESCRIPTION OF THE DISCLOSURE

With reference to the appended drawings, we describe a system for sharing visual data from a medical device. We describe in particular the sharing device used in the system, and the configuration of a remote server which provides a platform for communications between users in which the visual data from the medical device is shared. We also describe a computer-implemented method and associated software involved in providing this platform.

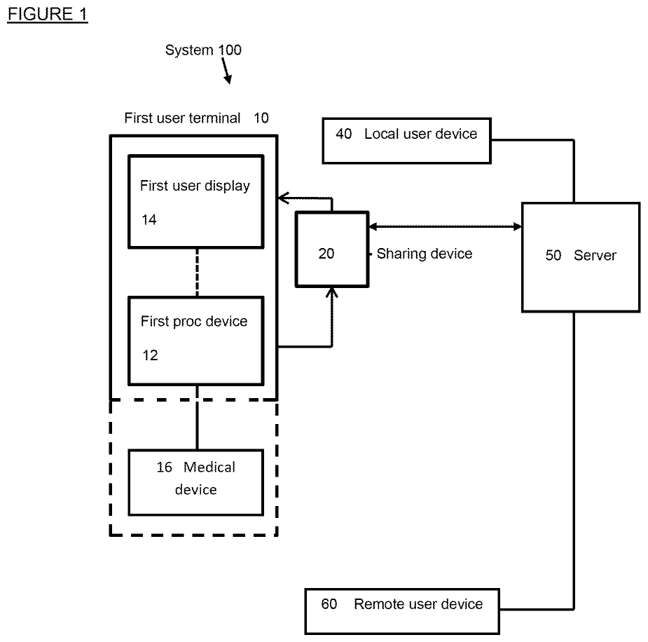

With reference to FIG. 1 of the drawings, a system 100 is illustrated. The system 100 as described is for sharing visual data from a medical device 16. In embodiments of the described technology, the medical device 16 is a medical scanning device producing visual imaging data as its output. For example, the medical device may be one of: a radiographic imaging device such as those used in fluoroscopy procedures or X-ray scanning procedures, a magnetic resonance imaging device (MRI scanner), a computerised tomography (CT) scanner, a positron emission tomography (PET) scanner, an ultrasounds scanner, a photoacoustic imaging scanner, a functional near-infrared (FNIR) spectroscopy scanner, or a magnetic particle imaging scanner. It should be understood that embodiments of the present disclosure are suitable for sharing any data having a visual representation; such as charts, graphs, 2D images, 3D models, or the like. The visual representation may include one or more images, or a stream of images in time sequence. In such embodiments, the data received from the medical device 16 includes medical device visual data.

In particular, the technology described is advantageous for the purpose of sharing images and models resulting from medical imaging processes, for the purpose of obtaining advice and expertise from an expert situated remotely from the scanning process. While it is envisaged that the medical device visual data may be obtained in a “live” setting, in which the scan is being conducted at the time the sharing takes place—and the image output of the scan is then shared remotely—the system is also suitable for sharing medical device visual data that has been obtained prior to the sharing taking place; stored as a data file and opened at the first user terminal to review the data. In other words, the step of obtaining the medical device visual data from a scan, and the step of obtaining medical device visual data at the first user terminal may not take place simultaneously or in quick succession.

With reference to FIGS. 1 to 3 of the drawings, in general terms, the system 100 provides a first user terminal 10 having a first processing device 12 configured to obtain data from a medical device 16. The first user terminal 10 provides the first processing device 12 and a first user display 14 configured to display the medical device visual data. The first user terminal 10 may include the medical device 16 (i.e., the actual scanning equipment), or else it may be connected to the medical device 16. Either way, medical device visual data is obtained from the medical device 16.

So, in other words, the first user terminal 10 may form the scanning unit itself—for example, the first user terminal 10 provides a first processing device 12, a first user display 14, and a medical device 16 (i.e., a scanner), all as one assembled unit, wherein the scanned data is provided to the first user display 14 via the first processing device 12. In such embodiments, the medical device visual data is generated by the first user terminal 10.

For example, an X-ray scanning unit may provide a scanning device (i.e., the medical device 16), connected to a first processing device 12 that receives unprocessed data from the scanner and stores and or otherwise processes that output into a form that can be readily understood—i.e., image data or the like, resembling the received raw scan data. Data is then displayed to a user of the scanning unit via the first user display 14, and may be communicated to one or more connected devices.

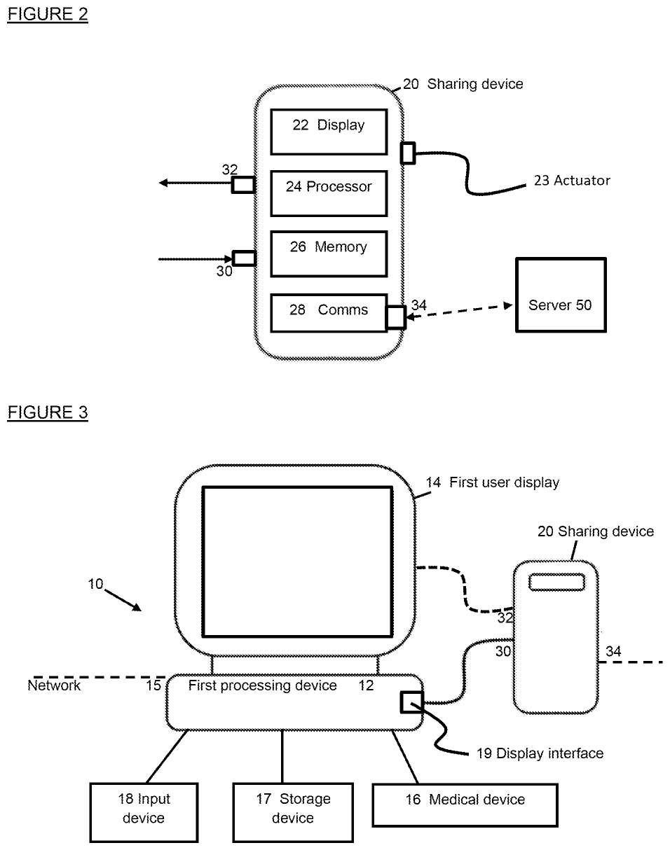

In another embodiment, the medical device 16 is provided as a standalone scanning unit, connected to a workstation or PC or the like, in which the case the first user terminal 10 is provided by that connected workstation. This layout is shown in FIG. 3, for example. In this embodiment, the first processing device 12 may be provided by a networked PC running an OS such as Windows™ for example, and the first user display 14 is provided by a monitor connected to a display interface of the first processing device 12. In this example, the first user terminal 10 receives the medical device visual data from the separate medical device 16.

In either of the above arrangements, the first processing device 12 may be configured to operate the medical device 16. For example, a user may initiate a scanning operation by controlling the medical device 16 via commands input using the first processing device 12.

A sharing device 20 is connected to the first user terminal 10, via a display interface 19 of the first processing device 12, to receive medical device visual data from the first user terminal 10. The sharing device provides a processor 24, memory device 26 and communication module 28. The sharing device typically provides a USB connector, for connection to a power supply or to a USB port of a computer for data transfer. The communication module 28 is configured to communicate with a server 50 over a remote connection 34. The sharing device 20 is configured to receive the medical device visual data from the first user terminal 10, and to communicate the medical device visual data to the server 50. The sharing device is configured to encrypt the medical device visual data, so that the medical device visual data sent to the server is encrypted. The encryption of the medical device visual data may be achieved using Advanced Encryption Standard (AES), with typical key sizes being 128, 192 or 256 bits. This encryption may be applied using Secure Sockets Layer (SSL) or Transport Layer Security (TLS), or Datagram Transport Layer Security (DTLS) for example, as is known in the art.

In some embodiments of the technology, the sharing device 20 provides a display 22 so that a user can determine a status of the device and/or view an operating condition of the device. For example, the display may provide one or more LEDs to indicate that the sharing device is connected to a power source, and/or may provide an LCD screen to display information such as a device identifier, an allocated device name, an error status, a ‘device ready’ status, a network connectivity status, or the like.

In some embodiments of the technology, the sharing device 20 provides a user-operable actuator 23 such as a push-button, switch, dial or the like. Using the actuator, a user may trigger the command to share the medical device visual data. This use of the actuator 23 to provide a command to share the medical device visual data may be used to provide a further layer of security to users of the system, requiring the user to be in the same location as the sharing device 20 in order to physically interact with the actuator 23 in order to allow sharing. In this way, the actuator does not in itself trigger the sharing, but is required once the command is provided to the sharing device 20 in order to enable the sharing to begin.

In embodiments of the technology, the sharing device 20 provides an input port 30 for receiving data from the first user terminal 10. More specifically (and as illustrated in FIG. 3), the first processing device 12 of the first user terminal 10 provides a display interface 19 for outputting data to a visual display such as the types of display port provided on most computers, including HDMI, DVI, VGA and DisplayPort technologies, for example. The input port 30 provides a corresponding input-so, for example, the input port may be an HDMI input so as to receive an HDMI output from the first user terminal 10. In this way, the sharing device 20 receives from the first user terminal 10 data that includes the medical device visual data. The medical device visual data may be only a portion of the data received by the sharing device 20. For example, the first processing device 12 of the first user terminal 10 may run an operating system such as Windows™ and within that operating system, an application may provide a view port to display the medical device visual data within the Windows™ interface, the visual representation of which is displayed to a user of the first user terminal 10 via its first user display 14. That display is itself connected to the first processing device 12 via a display interface 19. The sharing device 20 may receive the same data as that communicated to the first user display 14, a portion of which being the medical device visual data.

In embodiments of the technology, the first processing device 12 provides multiple display interfaces 19, so that the first user display 14 may be connected to the first processing device 12 via one display interface, and the sharing device 20 is connected to the first processing device 12 via another display interface. In the example embodiment illustrated in FIGS. 2 and 3, the sharing device 20 provides an output port 32 for outputting the data received at its input port 30. In such embodiments, the sharing device 20 effectively provides pass-through functionality such that the first user display 14 is connected to the output port 32 of the sharing device 20 so as to receive the data output from the first processing device 12 indirectly, via the sharing device 20. In such embodiments, the first processing device 12 may provide only a single display interface 19, which in turn is connected directly to the sharing device 20 and then indirectly to the first user display 14 via the output port of the sharing device 20.

The first processing device 12 is further connected to one or more input devices 18, such as a mouse and keyboard. The input devices 18 may include a microphone and/or a webcam, for enabling a user of the first user terminal 10 to communicate over a local network or over the internet using videotelephony or voice-over-internet protocol (VOIP) for example. The device may be connected to one or more speakers. In this way, the first processing device 12 may be suitable for videoconferencing, to allow a user to communicate with one or more remote users via audio and/or video links. The first processing device 12 may also provide, or be connected to, one or more storage devices 17 which may store medical device visual data, for example. The first processing device 12 is typically connected to a local network via a network port 15 such as a LAN-port, using an ethernet connection or the like. The first processing device 12 may provide in addition, or alternatively, a wireless (e.g. WiFi) network connection.

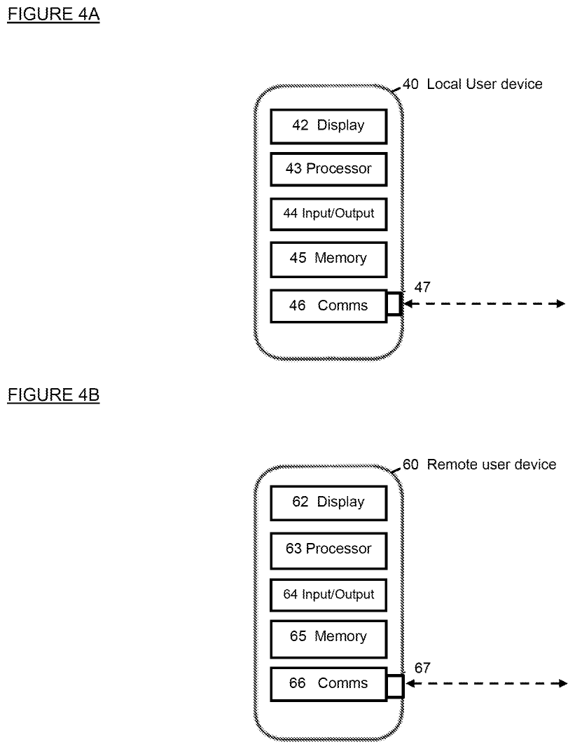

FIGS. 4A and 4B of the drawings illustrated respective local and remote user device 40, 60. In use of the system, a local user acts to obtain expertise or a second opinion from a remote user, to assess the medical device visual data. The local user operates a local user device 40, as shown in FIG. 4A. The local user device 40 may be a smartphone, a tablet computing device, a laptop, or a PC workstation, for example. The local device 40 typically provides a display 42, one or more processors 43, one or more input devices and/or output devices 44, which may include for example a mouse, a keyboard, a microphone, a camera and/or a speaker. The display 42 itself may provide touchscreen functionality acting as an input device 44. The local user device 40 may further include a memory device (i.e. RAM), storage (i.e., an SSD, hard disk, or similar). The local user device 40 includes a communications module 46, suitable for establishing a communication channel 47 with the server 50. For example, the communications module 46 provides one or more of: a local area network connection via an ethernet networking port; a data connection to a mobile data network using 5G, 4G or the like; and a WiFi connection to a local network.

Similarly, and with reference now to FIG. 4B, a remote user interacts with the system using a remote user device 60. The local and remote user devices 40, 60 have the same requirements and may both provide similar functionality. For example, the remote user device 60 may be a smartphone, a tablet computing device, a laptop, or a PC workstation, for example. The local device 60 typically provides a display 62, one or more processors 63, one or more input devices and/or output devices 64, which may include for example a mouse, a keyboard, a microphone, a camera and/or a speaker. The display 62 itself may provide touchscreen functionality acting as an input device 64. The local user device 60 may further include a memory device (i.e. RAM), storage (i.e., an SSD, hard disk, or similar). The local user device 60 includes a communications module 66, suitable for establishing a communication channel 67 with the server 50. For example, the communications module 66 provides one or more of: a local area network connection via an ethernet networking port; a data connection to a mobile data network using 5G, 4G or the like; and a WiFi connection to a local network.

Each of the local user device 40 and the remote user device 60 has access to a remote support application. The remote support application may be stored locally in storage on the local user device 40, or may alternatively be hosted on the server 50, or on another server accessible via the internet. For example, the remote support application may be hosted on a web server and accessed via a URL.

The remote support application allows the local user to initiate a communication channel 47 between the local user device 40 and the remote user device 60 via the server 50. To achieve this, the local user operates the remote support application and selects the remote user device 60 from a stored list of user devices. The stored data defining the user devices may include device identifier information associated with the user devices (which may include a phone number, email address, MAC, or IP address associated with the user device), a name of the user associated with the device, and/or a specialism of the user associated with the device, for example. This user device data is preferably stored at the server 50, but may in addition or alternatively be stored on the local user device.

Once the local user device 40 has communicated its intention to initiate a communication channel to the server 50, the server uses the device identifier information associated with the identified remote user to communicate with the remote user device 60. The remote user is alerted to the request to initiate communication with the local user, and operates the remote user device 60 to accept the request to join the communication channel. The local user and remote user are then able to communicate with each other via the established communication channel between the local user device 40 and remote user device 60.

The local user device 40 is further operable to initiate sharing of the medical device visual data by causing the server 50 to communicate the command to share the medical device visual data to the sharing device 20. This initiation of sharing medical device visual data may be triggered by an option selected at the first user device 40 within an interface provided by the remote support application. The server 50 subsequently communicates the command to the sharing device 20 via its communication module 28, causing the sharing device 20 to begin sending the medical device visual data via the remote connection 34 to the server 50. Prior to sending the medical device visual data to the server 50, the sharing device 20 may, in embodiments, require a user to operate the actuator 23 in order to verify the request, as mentioned above.

The server 50 receives the medical device visual data from the sharing device 20, and subsequently provides that medical device visual data to the remote user device 60. In embodiments of the technology the medical device visual data is provided to the remote user device 60 via the communication channel established between the local user device 40 and remote user device 60. In such embodiments, the medical device visual data may be provided to both the local user device 40 and the remote user device 60 via that communication channel.

For example, and with reference to FIG. 5, the communication channel may be provided via a communication channel interface 42a on the displays 42, 62 of the local and remote user devices 40, 60. The communication channel interface 42a provides a communication interface 42b in which the local and remote users may communicate with one another. This may include a video stream from the camera of one or both of the local and remote user devices 40, 60 and audio information captured by a microphone of each respective device (i.e., forming an audio/video conference). Alternatively or in addition, the communication interface 42b may include a text messaging interface to allow the two users to communicate via text messages. The medical device visual data 42c is provided within the interface to the users, so that the users can see and discuss the details of the medical device visual data.

In embodiments of the technology, where the medical device visual data is encrypted as it is sent to the server 50, the local user device 40 and/or remote user device 60 may be configured to decrypt the medical device visual data so as to allow it to be displayed via the interface. In other embodiments, the server is configured to decrypt the medical device visual data. The communication channel formed between the server 50 and the local user device 40 and remote user device 60 may itself be encrypted, and where the medical device visual information is shared via that communication channel, the medical device visual information may be subject to that applied encryption—in which case the data is encrypted before being provided to the respective user devices 40, 60.

In embodiments of the technology, the medical device visual data is processed at the server 50 prior to the data being communicated to one or more of the local user device 40 and remote user device 60. The medical device visual data may be processed to apply one or more filters to the images forming the visual data. An artificial intelligence module may be configured to run on the processor 52 of the server 50 (which may implement a neural network, for example) to modify the images forming the visual data in some way. For example, the contrast, saturation or lighting balance may be altered, features may be detected within the images and/or particular medical conditions highlighted via annotations of the images, for example. This modified medical device visual data may then be communicated to the local and/or remote user devices 40, 60.

In embodiments of the technology, the local user device 40 may be provided by the first user terminal 10. In this case, the first user display 14 provides the display of the first user device, the first processing device 12 provides the processor and memory of the first user device, and the attached input devices 18 provide the input functionality required by the first user device. In this way, the network port 15 of the first user terminal 10 may be used to communicate with the server 50, so as to initiate the communication channel.

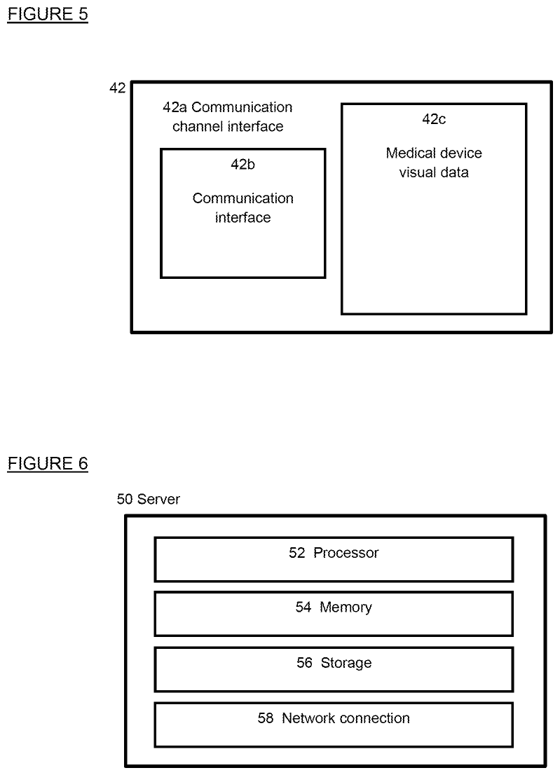

With reference to FIG. 6 of the drawings, the server 50 provides a processor 52, one or more memory devices 54, storage devices 56, and a communication module providing a network connection 58. The server 50 is preferably cloud-based and communicates with the local and remote user devices 40, 60 and sharing device 20 via the internet. By the term ‘server’, we mean a networked computing device configured to carry out the operations described.

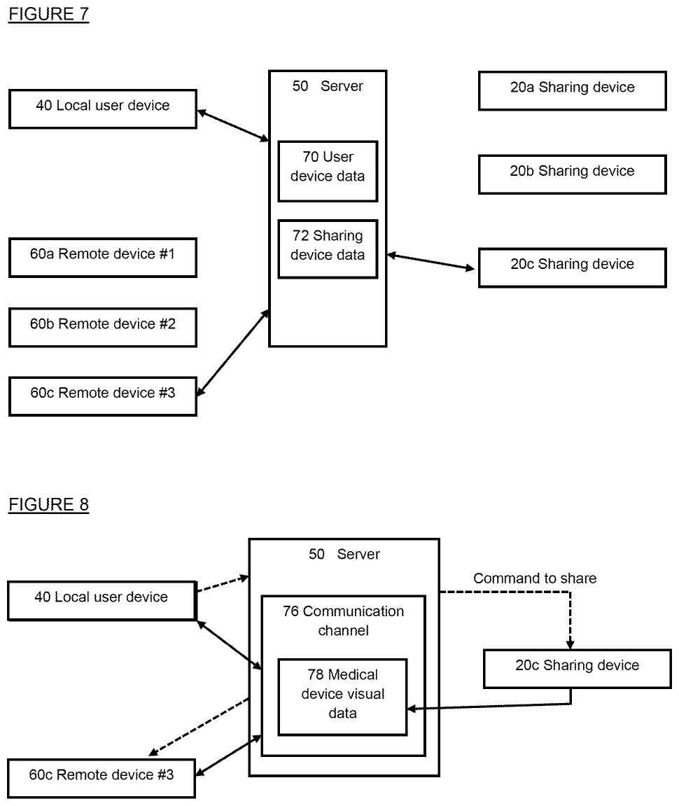

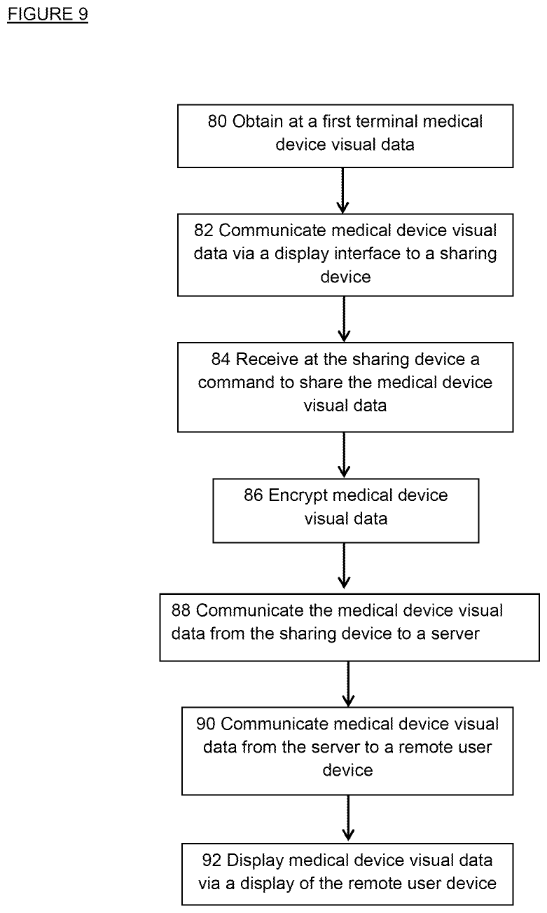

With reference to FIGS. 7 and 8, the server 50 stores data on its storage devices 56 including a sharing device data 72 providing records associating an identifier of each sharing device 20a, 20b, 20c with a corresponding communication address for that respective sharing device 20a, 20b, 20c. For example, the communication address may be an IP address identifying the sharing device 20a, 20b, 20c and allowing data to be sent to the sharing device 20a, 20b, 20c. The server 50 additionally stores user device data 70, providing a list of user devices 40, 60a, 60b, 60c as discussed above. The user device data 70 includes device identifier information associated with each user device (which may include a phone number, email address, MAC, or IP address associated with the user device), a device communication address (if the device identifier is not itself a communication address such as a phone number of IP address), a name of the user associated with the device, and/or a specialism of the user associated with the device, for example. It should be understood that the system 100 may include multiple sharing devices 20a, 20b, 20c, each connected to its own respective user terminal and associated medical device. In a system including three medical devices, a first user terminal 10, second user terminal and third user terminal may be provided, with associated first sharing device 20a, second sharing device 20b and third sharing device 20c. Each sharing device 20a, 20b, 20c is in communication with the server 50, and each is operable to share its respective medical device visual data.

We now describe the configuration of the server 50 and the operation of the system as a whole. The server 50 is configured to receive a communication request from the local user device 40, that request specifying another (different) one of the user devices 60a, 60b, 60c associated with a remote user. The server 50 is operable to identify in the stored user device data 70 a record associating the specific identified remote user device 60c, and to identify an associated device communication address, and to communicate with that remote user device 60c so as to initiate a communication channel between the local user device 40 and the specified remote user device 60c.

The server 50 is configured then to receive a request to share medical device visual data from the local user device 40. The local user selects the specified sharing device 20c they wish to share, out of multiple sharing devices 20a, 20b, 20c set up to operate within the system. The request specifies at least an identifier relating to that specific sharing device 20c, and in response to the request, the server 50 locates a record in its stored sharing device data 72. From this record, the server 50 determines a communication address for the identified sharing device 20c. The server subsequently uses the communication address of the sharing device 20c to communicate with that sharing device 20c, so as to provide the command to share the medical device visual data. In response, the sharing device 20c communicates the medical device visual data.

Using the system as described, the local user uses the remote support application on the local user device 40 to initiate a communication channel via the server 50 with a specific remote user, via their associated remote user device 60c. Once the channel is open, and the local user and remote user can communicate via that communication channel, the local user then initiates sharing of the medical device visual data by triggering the server 50 to send the command to the specified sharing device 20c to share its data. In response to that command, the sharing device 20c sends the medical device visual data to the server 50, and the server 50 incorporates that medical device visual data into the communication channel so that the remote user can see that visual data. The local and remote users are then able to analyse and discuss the medical device visual data.

In embodiments of the technology, the local user may require advice from the remote user about the output of multiple medical devices 16. It is possible, therefore, that once a communication channel with the remote user device 60c has been established, medical device visual data from multiple sharing devices 20a, 20b, 20c may be shared by the system within a single communication channel.

In this way, using the above example in which a local user has shared the medical device visual data of a first sharing device 20c, either the local user device 40 or the remote user device 60c may provide a further communication to the server 50 initiating sharing of data from a further sharing device 20b. For example, the remote user may determine that it would be beneficial to receive visual data from a second medical device associated with that further sharing device 20b, and so choose to trigger that sharing remotely. The server 50 then proceeds through the same steps as already outlined above-locating the relevant record associated with that further sharing device 20b, and communicating a command to it to share its data. The further sharing device 20b subsequently sends its medical device visual data to the server 50, which can then be communicated to the remote user device 60c via the communication channel. This may happen alongside the existing medical device visual data, so the two visual data streams can be compared side by side, for example. In other embodiments, the visual data may be overlaid.

In embodiments of the system, the local user and remote user may require input from another remote user. In that case, a request may be generated by either the local user device 40 or by the remote user device 60c to contact a further remote user device 60b, so as to join that remote user device 60b to the communication channel.

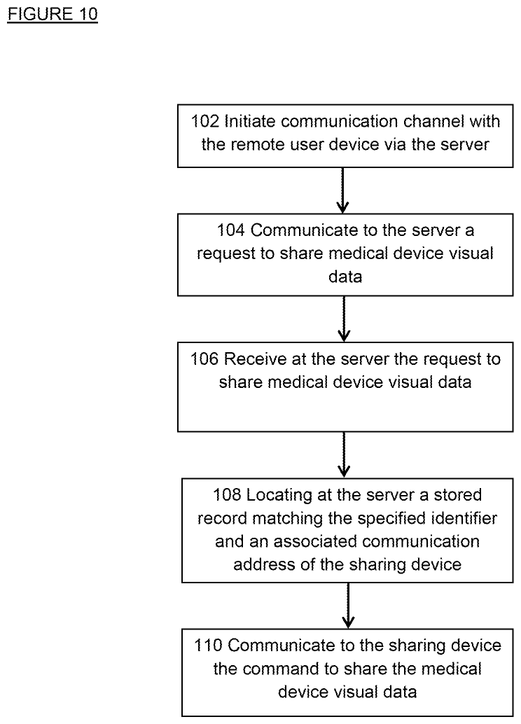

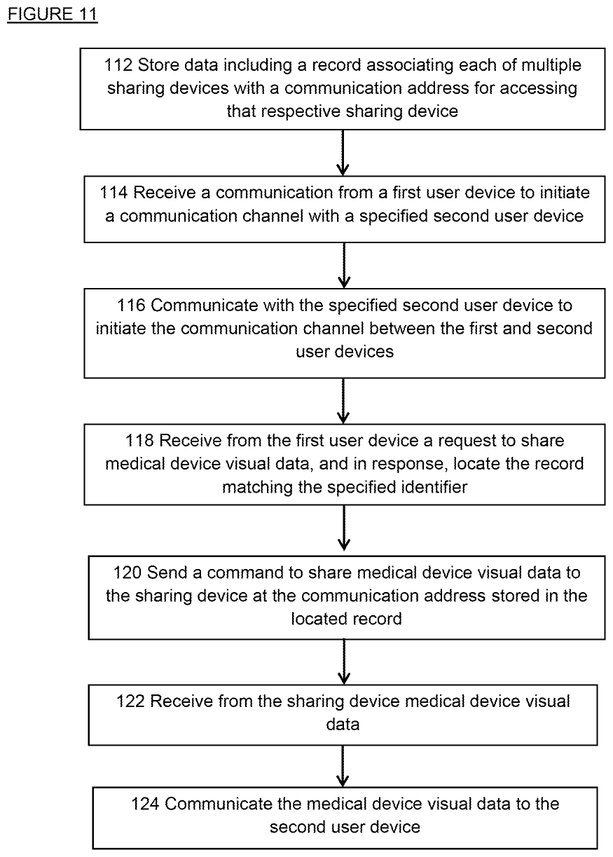

The method of operating the system can be summarised in the following broad steps, with reference to FIGS. 9 to 11.

Medical device visual data is obtained at the first user terminal 10, the data being either received from or generated by a medical device 16 at the first user terminal 10 (step 80). More specifically, the data is obtained by the first processing device 12. The medical device visual data is then communicated via the display interface 19 of the first processing device to a sharing device 20 (step 82). This may happen in parallel with communicating the same visual data to the first user display 14 for display to a user, or in series, with the sharing device 20 effectively providing a pass-through for the data to reach the first display device 14 via the sharing device 20 output port 32.

A local user operates the first user device 40 to initiate a communication channel with a remote user via their associated remote user device 60 (step 102). The local user identifies a sharing device 20 to be activated, and then identifies that sharing device 20 to the server 50 by sending a request to share medical device visual data, the request specifying an identifier relating to that specific sharing device 20 (step 104).

The local user then operates the local user device 40 to trigger the server 50 to send a command to the sharing device 20 share its medical device visual data (step 106). The server locates a stored record matching the specified identifier and an associated communication address of the sharing device 20 (step 108), and uses the communication address to communicate to the sharing device 20 the command to share the medical device visual data (step 110).

The sharing device 20 receives the command to share the medical device visual data, from the server 50 (step 84). In response to receiving that command, the sharing device 20 communicates the medical device visual data (which may be encrypted-step 86) from the sharing device 20 to the server 50 via its communication module 28 (step 88). The server 50 communicates the medical device visual data to the remote user device 60 (step 90), and subsequently that medical device visual data is displayed to the remote user via the display 62 of the remote user device 60 (step 92).

At the server 50 itself, the method of sharing visual data from the medical device involves the following steps. Data are stored at the server 50, each including a record associating an identifier of one of multiple sharing devices 20 with a communication address for accessing that respective sharing device 20 (step 112). On receipt of a communication from a first user device (i.e., the local user device 40) to initiate a communication channel with a specified second user device (i.e., the remote user device 60) (step 114), the server 50 sends a communication to the specified second user device to initiate the communication channel between the first and second user devices (step 116). Subsequently, the first user device sends a request to share medical device visual data, received at the server 50, the request specifying an identifier relating to one of the multiple sharing devices 20 (step 118). In response, the server 50 locates the record matching the specified identifier, and sends a command to share medical device visual data to the sharing device 20 at the communication address stored in the located record (step 120). In response, the sharing device 20 sends the data, which is received from the sharing device 20 at the server (step 122). Subsequently the medical device visual data is communicated to second user device (step 124).

A computer program is provided, including instructions which, when the program is executed on the processor 52 of the server 50, cause the server 50 to carry out the following steps. Multiple data are stored as sharing device data 72, each including a record associating an identifier of one of multiple sharing devices 20 with a communication address for accessing that respective sharing device 20. On receipt of a communication from a first user device to initiate a communication channel with a specified second user device, communicating with the specified second user device to initiate the communication channel between the first and second user devices. A request is received from the first user device, to share medical device visual data, the request specifying an identifier relating to one of the multiple sharing devices 20, and in response, locating the record matching the specified identifier. A command is sent from the server 50 to the identified sharing device 20 to share medical device visual data, using the communication address stored in the located record. Medical device visual data is received from the sharing device, and communicated to the second user device. Finally, a computer-readable medium is also provided, on which medium instructions are provided which, when executed by a computer, cause the computer to carry out the steps outlined above.

When used in this specification and claims, the terms “comprises” and “comprising” and variations thereof mean that the specified features, steps or integers are included. The terms are not to be interpreted to exclude the presence of other features, steps or components.

The disclosure may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the disclosure have been described, the scope of the appended claims is not intended to be limited solely to these embodiments. The claims are to be construed literally, purposively, and/or to encompass equivalents.

1. A system for sharing visual data from a medical device, the system including:
a first user terminal configured to obtain medical device visual data, the first user terminal providing a first user display configured to display the medical device visual data,
a sharing device connected to the first user terminal, and providing a communication module configured to communicate with a server, the sharing device being configured to receive the medical device visual data from the first user terminal, and
a remote user device providing a display, and a respective communication module configured to communicate with the server,
wherein the sharing device is operable to receive a command to share the medical device visual data, and in response to receiving that command, to communicate the medical device visual data to the server, and
wherein the remote user device is operable to receive a communication from the server including the medical device visual data, and to display the medical device visual data via its respective display.

2. The system of claim 1, wherein the sharing device is configured to encrypt the medical device visual data, and to communicate the encrypted medical device visual data to the server.

3. The system of claim 2, wherein the server is configured to communicate the encrypted medical device visual data to the remote user device, and the remote user device is configured to decrypt the medical device visual data.

4. The system of claim 1, wherein the medical device is a medical imaging device comprising one or more of: a radiographic imaging device, a magnetic resonance imaging device, a computerised tomography scanner, a positron emission tomography scanner, an ultrasound scanner, a photoacoustic imaging scanner, a functional near-infrared spectroscopy scanner, a magnetic particle imaging scanner.

5. The system of claim 1, wherein the first user terminal comprises a first processing device connected to the medical device for operating the medical device and receiving data from the medical device, the data including the medical device visual data, wherein the first processing device is connected to the sharing device via a display interface.

6. The system of claim 5, wherein the sharing device is connected to the first user display via a sharing device display interface, such that the medical device display data is routed to the first user display from the first processing device via the sharing device.

7. The system of claim 1, wherein the sharing device provides a user-operable actuator via which the command to share the medical device visual data is provided to the sharing device.

8. The system of claim 1, further including a local user device having a processor and communication module, the local user device being operable to initiate a communication channel with the remote user device via the server,
wherein the local user device is further operable to initiate sharing of the medical device visual data by causing the server to communicate the command to share the medical device visual data to the sharing device, and the server provides the medical device visual data to the remote user device.

9. The system of claim 1, wherein the first user terminal is operable to initiate a communication channel with the remote user device via the server,
wherein the first user terminal is further operable to initiate sharing of the medical device visual data by causing the server to communicate the command to share the medical device visual data to the sharing device, and the server provides the medical device visual data to the remote user device.

10. The system of claim 1, wherein:
the server is configured to store data including a record associating an identifier of the sharing device and a communication address of the sharing device,
the server being configured to receive a request to share the medical device visual data, the request specifying an identifier relating to the sharing device, and in response, to locate the record matching the specified identifier, and to communicate with the sharing device at the corresponding communication address so as to provide the command to share the medical device visual data.

11. A sharing device for use in the system of claim 1, the sharing device including:
a processor,
a port configured to receive medical device visual data from a display interface of a first user terminal, and
a communication module configured for communication with a server,
configured such that on receipt of a command to share the medical device visual data, the medical device visual data is communicated to the server.

12. The sharing device of claim 11, providing an encryption module configured to encrypt the medical device visual data, and the sharing device being configured to communicate the encrypted medical device visual data to the server.

13. The sharing device of claim 11, further including a mechanical actuator configured for operation by a user, such that operation of the actuator provides the command to share the medical device visual data.

14. The sharing device of claim 11, configured to receive the command to share the medical device visual data from the server via the communication module.

15. A computer-implemented method for sharing visual data from a medical device, the method including the steps of:
obtaining at a first terminal medical device visual data,
communicating the medical device visual data via a display interface to a sharing device,
receiving at the sharing device a command to share the medical device visual data,
in response to receiving that command, communicating the medical device visual data from the sharing device to a server,
communicating the medical device visual data, from the server to a remote user device, and
displaying the medical device visual data via a display of the remote user device.

16. The method of claim 15, further including encrypting the medical device visual data at the sharing device for communication to the server, and subsequently decrypting the encrypted medical device visual data at the remote user device.

17. The method of claim 15, further including the steps of:
at a local user device, initiating a communication channel with the remote user device via the server, and
at the local user device, communicating to the server a request to share medical device visual data, the request specifying an identifier relating to the sharing device,
receiving at the server the request to share the medical device visual data,
locating at the server a stored record matching the specified identifier and an associated communication address of the sharing device, and
using the communication address to communicate to the sharing device the command to share the medical device visual data.

18. A computer-implemented method for sharing visual data from a medical device, performed at a server, the method including the steps of:
storing multiple data each including a record associating an identifier of one of multiple sharing devices with a communication address for accessing that respective sharing device;
receiving a communication from a first user device to initiate a communication channel with a specified second user device,
communicating with the specified second user device to initiate the communication channel between the first and second user devices,
receiving from the first user device a request to share medical device visual data, the request specifying an identifier relating to one of the multiple sharing devices, and in response, locating the record matching the specified identifier,
sending a command to share medical device visual data to the sharing device at the communication address stored in the located record,
receiving from the sharing device medical device visual data, and
communicating the medical device visual data to the second user device.

19. A computer program providing instructions which, when the program is executed by a computer, cause the computer to carry out the steps of:
storing multiple data each including a record associating an identifier of one of multiple sharing devices with a communication address for accessing that respective sharing device;
receiving a communication from a first user device to initiate a communication channel with a specified second user device,
communicating with the specified second user device to initiate the communication channel between the first and second user devices,
receiving from the first user device a request to share medical device visual data, the request specifying an identifier relating to one of the multiple sharing devices, and in response, locating the record matching the specified identifier,
sending a command to share medical device visual data to the sharing device at the communication address stored in the located record,
receiving from the sharing device medical device visual data, and
communicating the medical device visual data to the second user device.
